(19) United States
(12) Patent Application Publication
(10) Pub. No.: US 2018/0117222 A1
(43) Pub. Date: May 3, 2018

(54) MEDICAL BIOABSORBABLE COMPOSITE HAVING FIBROUS CERAMIC REINFORCING AGENT AND METHOD FOR PREPARING THE SAME

(71) Applicant: OSTEONIC CO.,LTD., Seoul (KR)

(72) Inventors: MIN SU LEE, Cheongju-si (KR); HO JUN LEE, Cheongju-si (KR)

(73) Assignee: OSTEONIC CO.,LTD., Seoul (KR)

(21) Appl. No.: 15/678,114

(22) Filed: Aug. 16, 2017

(30) Foreign Application Priority Data
Oct. 31, 2016 (KR) 10-2016-0143441

(51) Int. Cl. A61L 31/14; A61L 27/58; A61L 31/12; A61L 27/46; A61L 27/56

(52) U.S. Cl. CPC A61L 31/148; A61L 27/58; A61F 2/3094; A61L 27/46; A61L 27/56; A61L 31/129

(57) ABSTRACT

The present invention relates to a medical bioabsorbable composite having a fibrous ceramic reinforcing agent and a method for preparing the same and, more particularly, to a medical bioabsorbable composite including a fibrous ceramic reinforcing agent including a fibrous ceramic reinforcing agent having a high aspect ratio, wherein the fibrous ceramic reinforcing agent may have a diameter ranging from 10 to 900 nm, and wherein a length to diameter aspect ratio may be equal to or greater than 5, and a method for preparing the same.

This application claims the benefit of the Korean Patent Application No. 10-2016-0143441, filed on Oct. 31, 2016, which is hereby incorporated by reference as if fully set forth herein.

### BACKGROUND OF THE INVENTION

### Field of the Invention

The present invention relates to a medical bioabsorbable composite having a fibrous ceramic reinforcing agent and a method for preparing the same and, most particularly, to a medical bioabsorbable composite having bioabsorbable properties and a method for preparing the same, wherein the reinforcing agent included in the composite corresponds to a fibrous ceramic reinforcing agent having a length to diameter aspect ratio equal to or greater than 5.

### Discussion of the Related Art

Medical implants and tools are being used as disposable orthopedic tools and surgical tools, such as pins, rods, nails, anchors, screws, plates, staplers, hooks, clips, and so on.

The related art medical implants and tools, which are manufactured of titanium, have been widely used. However, due to the absence of bioabsorbability, the usage of the related art medical implants and tools was disadvantageous in that they required a separate secondary surgery for removing the titanium implants and tools after the bones have been completely joined and fixed. In order to resolve such problems, diverse types of medical implants and tools, which are manufactured of bioabsorbable polymer, have been proposed.

However, in case of the medical implants and tools that are manufactured of bioabsorbable polymer, due to the limitations in the physical properties of the polymer, limitations in the application areas and applied products are inevitable. Accordingly, in order to overcome the above-described problems, the development of composites including inorganic particles or ceramic nanoparticles (or nanopowder) has been proposed.

Additionally, in the U.S. Pat. No. 4,843,112, a cross-linked bioabsorbable polymer is used as matrix, and bone cement dispersing calcium phosphate ceramic is proposed herein. Also, in the U.S. Pat. No. 4,604,097, orthopedic and/or dental implants formed (or fabricated) by using a bioabsorbable polymer composite that is reinforced by glass fiber are proposed.

And, the U.S. Pat. No. 5,338,772 proposes a porous composite used as implant material, wherein the matrix of the bioabsorbable polymer is reinforced by using calcium phosphate.

Moreover, in the U.S. Pat. No. 5,092,884, bioabsorbable polymer is used as matrix, and proposed herein is a composite that is prepared by using non-absorbable fiber.

Although the above-described related art bioabsorbable medical tools have the properties of being naturally absorbed in the human body, the related art bioabsorbable medical tools were disadvantageous in that they had low bending strength and low modulus of elasticity in bending.

Furthermore, since the problem of cytotoxicity still remains as a critical issue in the usage of nanoparticles, its application for medical usage still requires extensive verification to be carried out. And, therefore, the usage of nanoparticles is disadvantageous in that nanoparticles are not suitable for being used as a reinforcing agent for reinforcing physical properties.

### SUMMARY OF THE INVENTION

Accordingly, the present invention is directed to a bioabsorbable composite having fibrous ceramic reinforcing agents and a method for preparing the same that substantially obviate one or more problems due to limitations and disadvantages of the related art.

Another object of the present invention is to provide a medical bioasorbable composite having excellent bioabosorbability, bending strength, and also modulus of elasticity in bending.

Yet another object of the present invention is to provide a medical bioabsorbable composite having excellent bioabsorbability, bending strength, and modulus of elasticity in bending by providing ceramic nanopowder (or nanoparticles) having a diameter ranging from 10 to 900 nm and having a high length to diameter aspect ratio inside the composite.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, according to an exemplary embodiment of the present invention, provided herein is a medical bioabsorbable composite including a fibrous ceramic reinforcing agent may include a fibrous ceramic reinforcing agent having a high aspect ratio, wherein the fibrous ceramic reinforcing agent may have a diameter ranging from 10 to 900 nm, and wherein a length to diameter aspect ratio may be equal to or greater than 5.

Also, according to the present invention, the composite may be configured of the fibrous ceramic reinforcing agent and a bioabsorbable polymer.

Also, according to the present invention, a preparation of the fibrous ceramic reinforcing agent may be performed by mixing ceramic particles with a polymer, forming a nanofiber by performing an electrospinning process, and removing the polymer from the nanofiber by performing a burning process on the nanofiber.

Also, according to the present invention, the ceramic particles may correspond to one or more of a calcium phosphate compound including beta-tricalcium phosphate and hydroxyapatite (HA), Mg, Ni, and Cu, or an alloy of the above.

Also, according to the present invention, the bioabsorbable polymer may correspond to Polyglycolide, copolymers of Glycolide (or Glycolide copolymers), Glycolide-lactide copolymers, Glycolide-trimethylene carbonate copolymers, Polylactides, Poly-L-lactide, Poly-D-lactide, Poly-DL-lactide, L-lactide/DL-lactide copolymers, L-lactide/D-lactide copolymers, Polylactide copolymers, Lactide-trimethylene glycolide copolymers, Lactide-trimethylene carbonate copolymers, Lactide/δ-valerolactone copolymers, Lactide/ε-caprolactone copolymers, Polydepsipeptides(glycine-DL-lactide copolymer), Polylactide/ethylene oxide copolymers, Asymmetrically 3,6-substituted poly-1,4-dioxane-2,5-diones, Poly-β-hydroxybutyrate, Poly-β-hydroxybutyrate/β-hydroxyvalerate copolymers, Poly-β-hydroxypropionate, Poly-p-dioxanone, Poly-δ-valerolactone, Poly-ε-caprolactone, a copolymer of the above, or a mixture of the above.

Also, according to the present invention, a bioabsorbable polymer: fibrous ceramic reinforcing agent volume % ratio may correspond to 30 to 70 volume %: 70 to 30 volume %.

Also, according to the present invention, provided herein is a method for preparing a medical bioabsorbable composite including a fibrous ceramic reinforcing agent may include a mixing step mixing a polymer and ceramic nanopowder, an electrospinning step forming the mixed substances into a nanofabric structure by performing electrospinning, a burning step removing the polymer from the nanofiber so as to form the fibrous ceramic reinforcing agent, and a molding step performing compression molding after mixing the fibrous ceramic reinforcing agent with a bioabsorbable polymer.

Also, according to the present invention, in the mixing step, the mixing ratio between the polymer and the ceramic powder may be configured by setting a weight % of the ceramic nanopowder to 15 to 50 weight % of the total 100 weight %.

Also, according to the present invention, the fibrous ceramic reinforcing agent formed in the burning step may have a length to diameter aspect ratio equal to or greater than 5.

Also, according to the present invention, in the molding step, the medical bioabsorbable composite may be formed by performing compression molding. Herein, a pressure may be set to 50 to 200 MP, and a temperature may be set to 50 to 300° C.

Furthermore, according to the present invention, the method may further include a crystal growth step reinforcing connectivity of the ceramic reinforcing agent and enhancing physical properties, after the burning step.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 illustrates a preparation procedure of a composite according to an exemplary embodiment of the present invention.

FIG. 2 illustrates a mimetic diagram showing a preparation procedure of a fibrous ceramic reinforcing agent according to the exemplary embodiment of the present invention.

FIG. 3 illustrates a mimetic diagram showing a preparation procedure of a fibrous ceramic reinforcing agent including a step of crystal growth according to the exemplary embodiment of the present invention.

FIG. 4 illustrates a SEM image of the fibrous ceramic reinforcing agent according to the exemplary embodiment of the present invention.

### DETAILED DESCRIPTION OF THE INVENTION

Hereinafter, the preferred embodiments of the present invention be described in detail with reference to the accompanying drawings.

It should be understood that, wherever possible, the same reference numbers will be used throughout the appended drawings to refer to the same or like parts. In describing the present invention, in order to avoid and/or prevent any ambiguity or obscurity in the main concept of the present invention, some of the disclosed functions or structures will be omitted from the detailed description of the present invention.

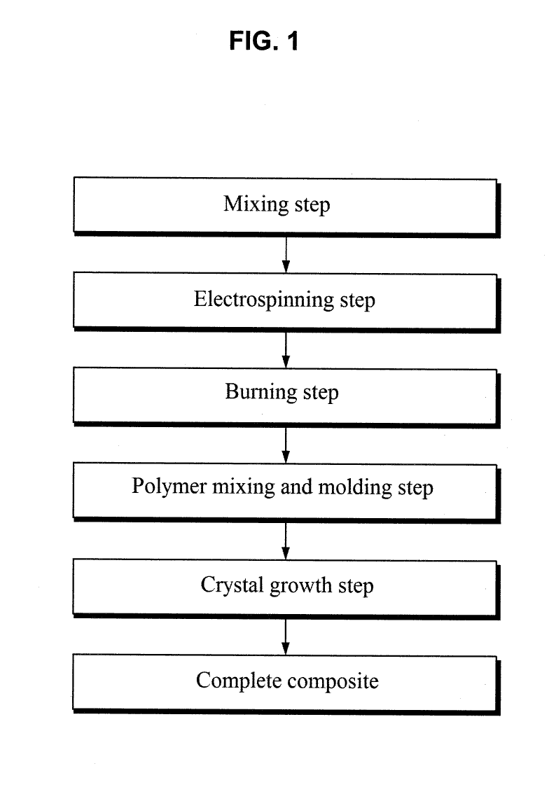

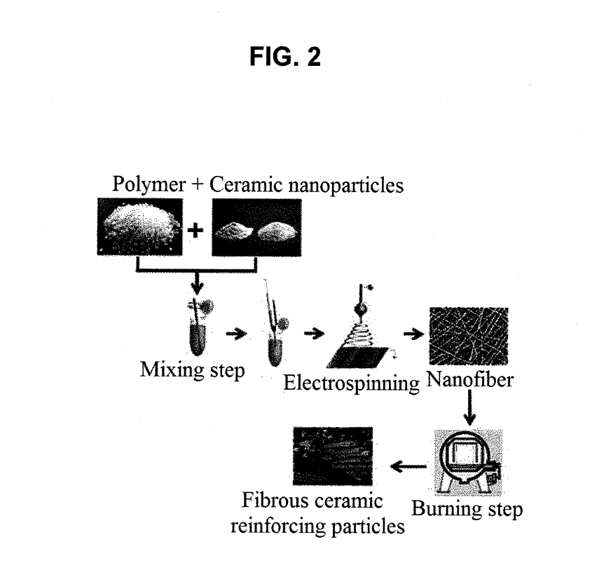

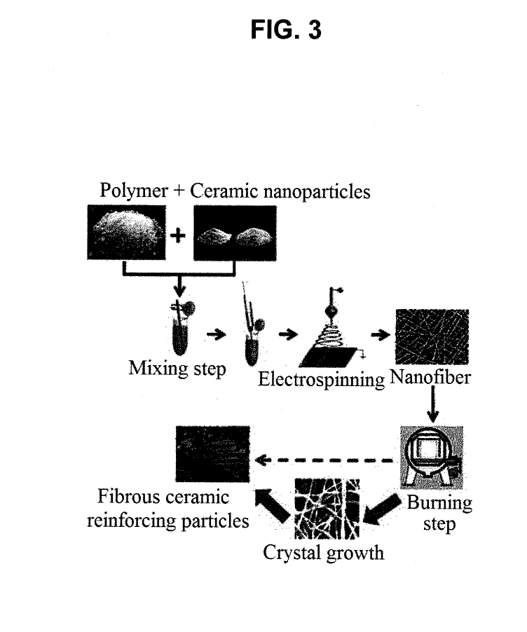

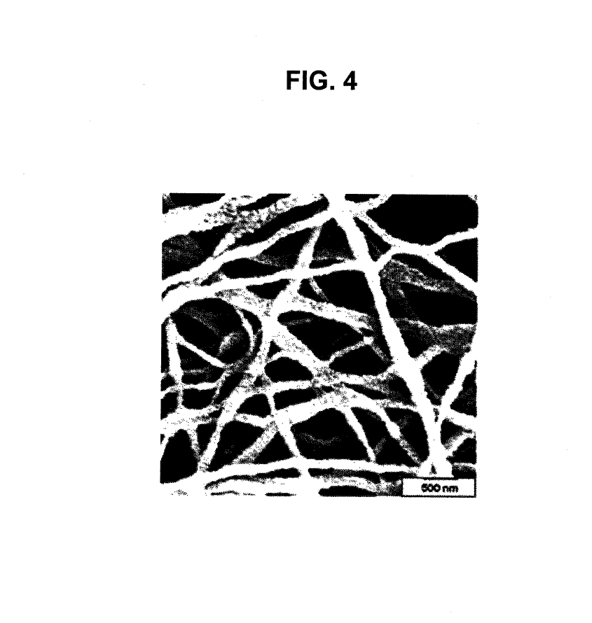

FIG. 1 illustrates a preparation procedure of a composite according to an exemplary embodiment of the present invention. FIG. 2 illustrates a mimetic diagram showing a preparation procedure of a fibrous ceramic reinforcing agent according to the exemplary embodiment of the present invention. FIG. 3 illustrates a mimetic diagram showing a preparation procedure of a fibrous ceramic reinforcing agent including a step of crystal growth according to the exemplary embodiment of the present invention. And, FIG. 4 illustrates a SEM image of the fibrous ceramic reinforcing agent according to the exemplary embodiment of the present invention.

According to the present invention, a medical bioabsorbable composite including a fibrous ceramic reinforcing agent may include a fibrous ceramic reinforcing agent having a high aspect ratio, wherein the fibrous ceramic reinforcing agent may have a diameter ranging from 10 to 900 nm, and wherein a length to diameter aspect ratio may be equal to or greater than 5. And, a method for preparing a medical bioabsorbable composite including a fibrous ceramic reinforcing agent may include a mixing step mixing a polymer and ceramic nanopowder, an electrospinning step forming the mixed substances into a nanofabric structure by performing electrospinning, a burning step removing the polymer from the nanofiber so as to form the fibrous ceramic reinforcing agent, and a molding step performing compression molding after mixing the fibrous ceramic reinforcing agent with a bioabsorbable polymer.

The method for preparing the reinforced bioabsorbable composite according to the present invention will hereinafter be described in detail.

The medical bioabsorbable composite may be prepared through a procedure of preparing the fibrous ceramic reinforcing agent (mixing step, electrospinning step, and burning step) and, then, mixing the preparation with a bioabsorbable polymer, and, then, molding the final mixture.

In order to prepare the fibrous ceramic reinforcing agent, polymer is dissolved in a solvent, and, then, the dissolved polymer is mixed with ceramic powder having the size of nanoparticles. At this point, the ceramic nanoparticles are equal to or less than approximately 500 nm. And, although the mixing ratio is not particularly limited, when mixing the polymer with ceramic powder, the weight of the ceramic powder may be set to 15 to 50 weight % (wt%) of the total 100 weight %. In case the electrospinning process is carried out by creating a mixture within the above-described range, a fibrous ceramic reinforcing agent having an aspect ratio of 5 or more may be prepared.

Ceramic nanosubstance may include one or more of a calcium phosphate compound including beta-tricalcium phosphate, hydroxyapatite (HA), and so on, a group configured of Mg, Ni, Cu, and so on, or may correspond to an alloy material of the same.

Since ceramic substance does not melt even at a high temperature, this substance is used as a constituent of nanofiber, which is formed by electrospinning. Accordingly, after removing the polymer by performing a burning process, a fibrous ceramic reinforcing agent, which is formed only of ceramic, may be prepared.

The polymer that is used for preparing the fibrous ceramic reinforcing agent is not particularly limited, and, therefore, any polymer that melts at a temperature ranging from 300 to 1000° C. may be used herein.

For example, non-absorbable polymers, such as PVC, PVA, and so on, and bioabsorbable polymers, such as PLA, PGA, PLLA, PLGA, PLDLA, PCL, PDO, and so on, may be used herein.

Diverse types of solvent, such as chloroform, chlorobenzene, methylene chloride, dichlorobenzene, trichlorobenzene, xylene, and so on, may be used.

The nanofiber having the size of nanoparticles may be formed (or prepared) by dissolving the polymer in the solvent and then mixing the ceramic powder in the dissolved polymer and, then, by performing electrospinning at a high pressure ranging from 8 to 10 kV in ambient temperature.

The nanofiber is formed to have a diameter ranging from 10 to 1000 nm, and the nanofiber may be formed to have a length to diameter aspect ratio that is equal to or greater than 5.

The nanofiber, which is formed by using the above-described process, may be treated with a burning process, so that only the ceramic substance remaining from the burning process can be used for the preparation of the fibrous ceramic reinforcing agent.

It will be preferable that the temperature of the burning process ranges from 300 to 1000° C. In the above-described temperature range, the ceramic substance remains without modification, and only the polymer is burnt and removed. Accordingly, since the ceramic substance remains without being meted at a high temperature, thereby allowing the fibrous ceramic reinforcing agent consisting only of ceramic material to be prepared.

Additionally, after the burning step, the present invention may further include a crystal growth step (see FIG. 3). By performing the crystal growth step, connections between the ceramic particles are strengthened, thereby allowing the volume or connecting parts of the fibrous ceramic structure to be reinforced. More specifically, the connection between the ceramic particles and the connectivity of the connecting parts of the fibrous ceramic structure are reinforced, and the physical properties may be enhanced.

The crystal growth step may include a dry crystal growth technique, wherein ceramic is melted or fused and then vacuum-vaporized by using highly heated plasma, or wherein ceramic is ionized and vacuum-vaporized by using RF magnetron, and a wet crystal growth technique, wherein the crystal growth process is carried out by adding a calcium chloride (CaCl2) solution and a Hexadecyl(cetyl) trimethyl ammonium bromide (CTAB) solution to an ammonium phosphate solution, which has a high hydrogen ion concentration.

After mixing the prepared fibrous ceramic reinforcing agent with the bioabsorbable composite, so that the fibrous ceramic reinforcing agent can become a constituent of the composite, the mixture is processed with compression molding, thereby forming (or fabricating) the medical bioabsorbable composite.

The bioabsorbable polymer may correspond to Polyglycolide, copolymers of Glycolide (or Glycolide copolymers), Glycolide-lactide copolymers, Glycolide-trimethylene carbonate copolymers, Polylactides, Poly-L-lactide, Poly-D-Iactide, Poly-DL-lactide, L-lactide/DL-lactide copolymers, L-lactide/D-lactide copolymers, Polylactide copolymers, Lactide-trimethylene glycolide copolymers, Lactide-trimethylene carbonate copolymers, Lactide/δ-valerolactone copolymers, Lactide/ε-caprolactone copolymers, Polydepsipeptides(glycine-DL-lactide copolymer), Polylactide/ethylene oxide copolymers, Asymmetrically 3,6-substituted poly-1,4-dioxane-2,5-diones, Poly-β-hydroxybutyrate, Poly-β-hydroxybutyrate/β-hydroxyvalerate copolymers, Poly-β-hydroxypropionate, Poly-p-dioxanone, Poly-δ-valerolactone, Poly-c-caprolactone, a copolymer of the above, or a mixture of the above.

It will be preferable that a bioabsorbable polymer: fibrous ceramic reinforcing agent volume % ratio corresponds to 30 to 70 volume %: 70 to 30 volume %. Within this range, the composite has excellent bioabsorbability, bending strength, and modulus of elasticity in bending.

A solvent may be used for the mixture of the bioabsorbable polymer and the fibrous ceramic reinforcing agent. Herein, the same solvent that was used for preparing the fibrous ceramic reinforcing agent may be used as the solvent for preparing the mixture, and diverse types of solvent, such as chloroform, chlorobenzene, methylene chloride, dichlorobenzene, trichlorobenzene, xylene, and so on, may be used.

The composite may be formed by mixing the fibrous ceramic reinforcing agent and the bioabsorbable polymer and, then, by processing the mixture with compression molding. The compression mold may be formed by using a compression molding machine. Herein, when performing the compression molding process, it will be preferable that the pressure ranges from 50 to 200 MPa and that the temperature ranges from 50 to 300° C.

Since the medical bioabsorbable composite according to the present invention is prepared with bioabsorbable polymers, the composite has bioabsorbable properties. Also, since a fibrous ceramic reinforcing agent is included in the medical bioabsorbable composite, the composite has excellent bending strength and modulus of elasticity in bending.

Additionally, as compared to the human bone, which has a bending strength ranging from 80 MPa to 120 MPa, and steel, which has a banding strength of approximately 280 MPa, the bending strength of the composite according to the present invention is equal to or greater than 290 MPa. Furthermore, as compared to the human bone, which has a modulus of elasticity in banding ranging from 10 to 17 GPa, the modulus of elasticity in bending of the composite according to the present invention is equal to or greater than 17 GPa.

As a result, the medical bioabsorbable composite according to the present invention may be molded to diverse forms and shapes, such as clamps, hooks, rods, pins, and so on, in order to be used as prosthetic tools for joining hard tissue and soft tissue, surgical implants, and so on.

Hereinafter, the exemplary embodiments of the present invention will be described in detail.

### First Embodiment

Preparation of the Fibrous Ceramic Reinforcing Agent

In order to prepare the fibrous ceramic reinforcing agent, a polymer, such as polyvinyl alcohol (PVA) is dissolved in a methylene chloride solvent. Thereafter, ceramic powder having the size of nanoparticles is mixed to the dissolved PVA. For the polymer to ceramic powder mixing ratio of the mixture, the weight of the ceramic powder is set to 30 weight %.

Hydroxyapatite (HA) having the size of 20 nm is used as the ceramic nanopowder. And, by performing electrospinning on the ceramic nanopowder under a high pressure of 10 kV, nanofiber having an average diameter of 200 nanometers (nm) is formed. The nanofiber, which is formed by electrospinning is configured of a mixed structure of polymer and ceramic. Subsequently, the polymer is removed from the nanofiber, which is configured of a mixture of polymer and ceramic, by performing a burning process on the nanofiber at a temperature of 500° C.

The fibrous structure having its polymer removed by being processed with burning is ground by using a ball mill and, then, dried for a period of 12 hours in a vacuum state in liquid nitrogen at a temperature of 120° C., thereby forming a fibrous ceramic reinforcing agent. The formed fibrous ceramic reinforcing agent corresponds to a fibrous material having a length to diameter aspect ratio that is equal to or greater than 5.

Preparation of the Composite

The formed fibrous ceramic reinforcing agent is mixed with polyglycolide, which is a bioabsorbable polymer. And, then, the composite is prepared by processing the mixture with compression molding by using a compression molding machine. More specifically, the composite is prepared by applying heat and performing extrusion molding and compression molding on the mixture of fibrous ceramic reinforcing agent and bioabsorbable polymer. Herein, the medical composite may be formed at a temperature of 180° C. under a pressure of 100 MPa.

### Second Embodiment

The preparation method is similar to the first embodiment (Embodiment 1).

Herein, however, the fibrous ceramic reinforcing agent is formed by dissolving the polymer PVC and by using Mg as the nanopowder. By performing burning at a temperature of 800° C., a fibrous ceramic reinforcing agent having its polymer removed therefrom is formed.

Furthermore, during the preparation of the composite, a medical bioabsorbable composite is formed at a temperature of 180° C. under a pressure of 150 MPa.

### Third Embodiment

The preparation method is similar to the first embodiment (Embodiment 1).

Herein, however, the fibrous ceramic reinforcing agent is formed by dissolving the polymer PGA and by using beta-tricalcium phosphate as the nanopowder. By performing burning at a temperature of 500° C., a fibrous ceramic reinforcing agent having its polymer removed therefrom is formed.

Furthermore, during the preparation of the composite, a medical bioabsorbable composite is formed at a temperature of 180° C. under a pressure of 150 MPa.

### Fourth Embodiment

The preparation method is similar to the first embodiment (Embodiment 1).

Herein, however, the fibrous ceramic reinforcing agent is formed by dissolving the polymer PLLA and by using Ni as the nanopowder. By performing burning at a temperature of 1000° C., a fibrous ceramic reinforcing agent having its polymer removed therefrom is formed.

Additionally, a crystal growth step is added after the burning step. Herein, the dry crystal growth technique, wherein highly heated plasma is used to melt and fuse the ceramic material and then to perform vacuum evaporation.

Furthermore, during the preparation of the composite, a medical bioabsorbable composite is formed at a temperature of 180° C. under a pressure of 150 MPa.

Results of a physical property test carried out by using the composites prepared according to the first exemplary embodiment to the fourth exemplary embodiment of the present invention are as shown below in Table 1.

TABLE 1

| | | Modulus of Elasticity |
|---|---|---|
| | Bending Strength (MPa) | in bending (GPa) |
| Embodiment 1 | 295 | 18 |
| Embodiment 2 | 300 | 20 |
| Embodiment 3 | 307 | 18 |
| Embodiment 4 | 303 | 19 |

As described above, the medical bioabsorbable composite having a fibrous ceramic reinforcing agent and the method for preparing the same have the following advantages. Since the reinforced medical bioabsorbable composite is prepared with bioabsorbable polymers, the composite has bioabsorbable properties. Also, since an ultra-thin micro-fibrous ceramic reinforcing agent is included in the medical bioabsorbable composite, the composite has excellent bending strength and modulus of elasticity in bending.

Also, according to the present invention, as compared to the human bone, which has a bending strength ranging from 80 MPa to 120 MPa, and steel, which has a banding strength of approximately 280 MPa, the bending strength of the composite according to the present invention is equal to or greater than 290 MPa. Furthermore, as compared to the human bone, which has a modulus of elasticity in banding ranging from 10 to 17 GPa, the modulus of elasticity in bending of the composite according to the present invention is equal to or greater than 17 GPa.

Also, the medical bioabsorbable composite according to the present invention may be molded to diverse forms and shapes, such as clamps, hooks, rods, pins, and so on, in order to be used as prosthetic tools for joining hard tissue and soft tissue, surgical implants, and so on.

Although the present invention has been described according to the preferred exemplary embodiment of the present invention, it will be apparent to those skilled in the art that various modifications and variations can be made in this specification without departing from the spirit or scope of this specification.

Thus, it is intended that this specification covers the modifications and variations of this invention provided they come within the scope of the appended claims and their equivalents. It is also apparent that such variations of this specification are not to be understood individually or separately from the technical scope or spirit of this specification, and all differences lying within the scope of the appended claims and their equivalents should be interpreted as being included in the present invention.

What is claimed is:

1. A medical bioabsorbable composite including a fibrous ceramic reinforcing agent, comprising:
a fibrous ceramic reinforcing agent having a high aspect ratio,
wherein the fibrous ceramic reinforcing agent has a diameter ranging from 10 to 900 nm,
wherein a length to diameter aspect ratio is equal to or greater than 5, and
wherein a preparation of the fibrous ceramic reinforcing agent is performed by mixing ceramic particles with a polymer, forming a nanofiber by performing an electrospinning process, and removing the polymer from the nanofiber by performing a burning process on the nanofiber.

2. The medical bioabsorbable composite of claim 1, wherein the composite is configured of the fibrous ceramic reinforcing agent and a bioabsorbable polymer.

3. The medical bioabsorbable composite of claim 1, wherein the ceramic particles correspond to one or more of a calcium phosphate compound including beta-tricalcium phosphate and hydroxyapatite (HA), Mg, Ni, and Cu, or an alloy of the above.

4. The medical bioabsorbable composite of claim 1, wherein the bioabsorbable polymer corresponds to Polyglycolide, copolymers of Glycolide (or Glycolide copolymers), Glycolide-lactide copolymers, Glycolide-trimethylene carbonate copolymers, Polylactides, Poly-L-lactide, Poly-D-lactide, Poly-DL-lactide, L-lactide/DL-lactide copolymers, L-lactide/D-lactide copolymers, Polylactide copolymers, Lactide-trimethylene glycolide copolymers, Lactide-trimethylene carbonate copolymers, Lactide/δ-valerolactone copolymers, Lactide/ε-caprolactone copolymers, Polydepsipeptides(glycine-DL-lactide copolymer), Polylactide/ethylene oxide copolymers, Asymmetrically 3,6-substituted poly-1,4-dioxane-2,5-diones, Poly-β-hydroxybutyrate, Poly-β-hydroxybutyrate/β-hydroxyvalerate copolymers, Poly-β-hydroxypropionate, Poly-p-dioxanone, Poly-δ-valerolactone, Poly-ε-caprolactone, a copolymer of the above, or a mixture of the above.

5. The medical bioabsorbable composite of claim 1, wherein a bioabsorbable polymer: fibrous ceramic reinforcing agent volume % ratio corresponds to 30 to 70 volume %: 70 to 30 volume %.

6. A method for preparing a medical bioabsorbable composite including a fibrous ceramic reinforcing agent, comprising:
a mixing step mixing a polymer and ceramic nanopowder;
an electrospinning step forming the mixed substances into a nanofabric structure by performing electrospinning;
a burning step removing the polymer from the nanofiber so as to form the fibrous ceramic reinforcing agent; and
a molding step performing compression molding after mixing the fibrous ceramic reinforcing agent with a bioabsorbable polymer.

7. The method of claim 6, wherein, in the mixing step, the mixing ratio between the polymer and the ceramic powder is configured by setting a weight % of the ceramic nanopowder to 15 to 50 weight % of the total 100 weight %.

8. The method of claim 6, wherein the fibrous ceramic reinforcing agent formed in the burning step has a length to diameter aspect ratio equal to or greater than 5.

9. The method of claim 6, wherein, in the molding step, the medical bioabsorbable composite is formed by performing compression molding, and
wherein a pressure is set to 50 to 200 MP, and a temperature is set to 50 to 300° C.

10. The method of claim 6, further comprising:
a crystal growth step reinforcing connectivity of the ceramic reinforcing agent and enhancing physical properties, after the burning step.
